(19) United States
(12) Patent Application Publication
(10) Pub. No.: US 2019/0314266 A1
(43) Pub. Date: Oct. 17, 2019

(54) HAIR TREATMENT COMPOSITIONS COMPRISING A HOMOPOLYMER OF (3 -ACRYLAMIDOPROPYL) TRIMETHYL AMMONIUM CHLORIDE

(71) Applicant: Conopco, Inc., d/b/a UNILEVER, Englewood Cliffs, NJ (US)

(72) Inventors: Ningning LI, Bebington, Wirral (GB); Arash Mohajer MOGHADAM, Bromborough, Wirral (GB); Joseph MUSCAT, Warrington (GB); Susan PYE, Wirral (GB); Cheryl Anne TAYLOR, Bromborough, Wirral (GB)

(21) Appl. No.: 16/343,100

(22) PCT Filed: Nov. 7, 2017
(86) PCT No.: PCT/EP2017/078507

§ 371 Date: Apr. 18, 2019

(30) Foreign Application Priority Data
Nov. 9, 2016 (EP) 16198025.5

(51) Int. Cl. A61K 8/88; A61K 8/92; A61K 8/365; A61Q 5/00; A61Q 5/12

(52) U.S. Cl. CPC A61K 8/88; A61K 8/92; A61Q 5/12; A61Q 5/002; A61K 8/365

(57) ABSTRACT

The invention provides a hair treatment composition for the strengthening and/or repair of damaged hair, the composition comprising, in an aqueous continuous phase: (i) one or more oily liquid hair conditioning agents selected from solubilized oily liquid hydrocarbons and mixtures thereof; (ii) a hair substantive cationic conditioning polymer which is a homopolymer of (3-acry-lamidopropyl) trimethyl ammonium chloride, and (iii) a one or more aliphatic carboxylic acids having a molecular weight of 175 g/mol or less, which is a tartaric acid.

### FIELD OF THE INVENTION

The present invention relates to hair treatment compositions. In particular, the invention relates to hair treatment compositions which comprise a blend of fibre actives for the strengthening and/or repair of damaged hair.

### BACKGROUND OF THE INVENTION

The hair fibre is susceptible to damage from a number of sources. These include environmental factors such as excessive exposure to UV; chemical factors such as bleaching or other oxidative processes; and mechanical factors such as overuse of heated styling appliances.

Since the hair fibre is a multilayer structure, once hair is damaged, repair treatments must intervene on several levels to repair it—from the middle of the core to the surface of the cuticle.

Film-forming polymers are often used in hair damage repair treatments because they alter hair surface properties, imparting smoothing and gliding effects and shine, and have a significant impact on the macroscopic behaviour of the hair array. However, film-forming polymers are by nature designed to provide hair fibres with a hydrophobic coating that may slow or prevent the penetration of actives. Therefore such treatments may not provide multilayer repair benefits to the fibre.

The present invention addresses this problem.

### SUMMARY OF THE INVENTION

The present invention provides a hair treatment composition for the strengthening and/or repair of damaged hair, the composition comprising, in an aqueous continuous phase:

(i) one or more oily liquid hair conditioning agents selected from solubilized oily liquid hydrocarbons and mixtures thereof;

(ii) a hair substantive cationic conditioning polymer which is a homopolymer of (3-acrylamidopropyl) trimethyl ammonium chloride, and

(iii) A aliphatic carboxylic acids having a molecular weight of 175 g/mol or less, which is a tartaric acid.

### DETAILED DESCRIPTION AND PREFERRED EMBODIMENTS

The composition of the invention includes one or more oily liquid hair conditioning agents (ii) selected from solubilized oily liquid hydrocarbons and mixtures thereof.

For the purposes of the present invention, the term “oil” means a non-aqueous compound which is immiscible with water (distilled or equivalent) at a concentration of 0.1 wt %, at 25° C. The term “oily liquid” means an oil that is capable of flowing under its own weight under ambient conditions (1 atmosphere, 25° C.).

Oily liquid hydrocarbons suitable for use in the invention will generally have a kinematic viscosity at 40° C. of 1000 cS (mm²·s⁻¹) or less, preferably 500 cS (mm²·s⁻¹) or less, more preferably 50 cS (mm²·s⁻¹) or less, and most preferably 10 cS (mm²·s⁻¹) or less, such as from 0.5 to 10 cS (mm²·s⁻¹).

Examples of such materials include C₄-C₅₀ straight or branched chain, saturated or unsaturated aliphatic or cycloaliphatic hydrocarbons and mixtures thereof. Straight chain hydrocarbons will preferably contain from about 12 to about 30 carbon atoms. Branched chain hydrocarbons can and typically may contain higher numbers of carbon atoms. Also suitable are polymeric hydrocarbons, such as polymers of C₂₋₆ alkenyl monomers (e.g. polyisobutene, polybutene) and poly⋅-olefin oils derived from 1-alkene monomers having from about 6 to about 16 carbons, preferably from about 6 to about 12 carbon atoms (e.g. polymers derived from 1-octene, 1-decene, 1-dodecene, 1- tetradecene, 1-hexadecene, and mixtures thereof). Polymeric hydrocarbons for use in the invention can be straight or branched chain polymers, and may be hydrogenated. The number average molecular weight of such polymeric materials can vary widely, but will typically range from about 200 up to about 3000.

Preferred oily liquid hydrocarbons for use in the invention include mineral oils. The term “mineral oil” in the context of this invention generally denotes an oily liquid mixture of saturated hydrocarbons with boiling points greater than 200° C., and which is obtained from petroleum (i.e. a mineral source). Mineral oil saturated hydrocarbons include straight chain (paraffinic), branched chain (isoparaffinic) and cyclic (naphthenic) structures, and molecules containing all three configurations, with the number of carbon atoms per hydrocarbon molecule generally ranging from about C₁₅ to about C₅₀. Mineral oils suitable for use in the invention are typically obtained from petroleum through various refining steps (e.g. distillation, extraction and/or crystallisation) and subsequent purification (e.g. acid treatment and/or catalytic hydrotreatment).

Mineral oils may also be characterised in terms of their viscosity. “Light” mineral oils will generally have a kinematic viscosity of about 34 cS (mm²·s⁻¹) or less at 40° C. and “heavy” mineral oils will generally have a kinematic viscosity ranging from about 35 cS (mm²·s⁻¹) up to about 240 cS (mm²·s⁻¹) at 40° C.

Light mineral oils (as defined above) are preferred for use in the invention. More preferably such light mineral oils have a kinematic viscosity of about 10 cS (mm²·s⁻¹) or less at 40° C. Most preferably the kinematic viscosity ranges from about 3 to about 5 cS (mm²·s⁻¹) at 40° C. Materials of this type are commercially available from Sonneborn Inc. under the brand name Lytol®.

Mixtures of any of the above-described materials may also be used.

The level of oily liquid conditioning agent (i) in compositions of the invention depends on the particular material (s) used, but generally ranges from about 0.5 to about 3% by weight based on the total weight of the composition.

In a preferred composition according to the invention the oily liquid conditioning agent (i) is light mineral oil (as defined above), at a level ranging from about 0.45 to about 2%, more preferably from about 0.5 to about 1.5% (by weight based on the total weight of the composition).

In a typical composition according to the invention, the oily liquid conditioning agent (i) is solubilised in wormlike micelles in the aqueous continuous phase to form a micro emulsion which is stable to phase separation.

“Wormlike micelles” in the context of this invention are elongated and flexible aggregates formed by the self-assembly of surfactant molecules in water. Above a threshold concentration, wormlike micelles entangle into a transient network, reminiscent of polymer solutions, and display viscoelastic properties. However, unlike a covalently bonded polymer backbone, the micelles are in a state of thermodynamic equilibrium with the solvent and are perpetually broken and reformed under Brownian fluctuations. This leads to a broad and dynamic distribution of micelle lengths which can change under an imposed shear or extensional flow.

Wormlike micelles can be fully described by a number of structural parameters, which cover a broad range of length-scales. The overall length of the micelles is referred to as the contour length L and varies between a few (e.g. about 1 to 10) nanometers up to a few (e.g. about 1 or 2) microns. Cryo-TEM provides a direct visualization of the micelles and can be used to estimate the contour length, while light and neutron scattering give a more accurate determination. Radii of wormlike micelles are typically a few (e.g. about 1 to 10) nm. Another key structural parameter in the description of wormlike micelles is the persistence length /ₚ, the length over which the micelles are considered rigid. Although wormlike micelles can be extremely flexible and micrometres long, their large cross-section implies that on smaller length-scales (of order /ₚ) they act as rigid rods. Techniques such as rheology, light and neutron scattering and flow birefringence have been employed to estimate /ₚ, as well as simulations. Experimentally, persistence lengths from about 10 to about 40 nm have been reported in neutral systems. For charged wormlike micelles, the persistence length varies significantly with surfactant structure, counter-ion and salt concentration, but is typically a few tens of nanometers (e.g. about 30 to about 100 nm).

Preferably the oily liquid conditioning agent (i) is solubilised in the aqueous continuous phase via the incorporation of at least one inorganic electrolyte and at least one linker molecule. “Linker molecules” in the context of this invention are chemical additives used in surfactant systems that enhance the surfactant-oil or surfactant-water interactions. Lipophilic linkers segregate near the oil side of the interface close to the tails of the surfactants. The presence of the lipophilic linker extends the impact of the surfactant deeper into the oil phase and may promote additional orientation of the oil molecules. Hydrophilic linkers are surfactant-like molecules that coadsorb with the surfactant at the oil/water interface, but have a minimal interaction with the oil molecules. The adsorption of the hydrophilic linker at the oil/water interface increases the total interfacial area. The term “inorganic electrolyte” in the context of this invention denotes an inorganic salt which dissolves in water and ionizes but whose ions do not aggregate in solution as, for example, do the ions of a surface active agent which aggregate to form micelles.

Suitable linker molecules for use in the invention include benzoic acid, caprylic acid (and/or their sodium or potassium salts) and mixtures thereof.

When included, the level of linker molecule preferably ranges from about 0.01 to about 1%, more preferably from about 0.02 to about 0.5% (by weight based on the total weight of the composition).

Suitable inorganic electrolytes for use in the invention include metal chlorides (such as sodium chloride, potassium chloride, calcium chloride, magnesium chloride, zinc chloride, ferric chloride and aluminium chloride) and metal sulfates (such as sodium sulfate and magnesium sulfate). The inorganic electrolyte is used to assist in the solubilisation of the hydrocarbon-based oily liquid conditioning agents (ii) and to provide viscosity to the composition.

Examples of preferred inorganic electrolytes for use in the invention include sodium chloride, potassium chloride, magnesium sulfate and mixtures thereof.

Mixtures of any of the above-described materials may also be suitable.

When included, the level of inorganic electrolyte in compositions of the invention generally ranges from about 1 to about 25%, preferably from about 1.5 to about 20% (by total weight inorganic electrolyte based on the total weight of the composition).

A composition of the invention may suitably have a viscosity ranging from 3,000 to 10,000 mPa·s, preferably from 4,000 to 9,000 mPa·s and more preferably from 4,000 to 6,000 mPa·s, when measured using a Brookfield V2 viscometer (spindle RTV5, 1 minute, 20 rpm) at 30° C.

The composition of the invention includes a hair substantive cationic conditioning polymer (ii) which is a homopolymer of (3-acrylamidopropyl) trimethyl ammonium chloride.

WO2013/122861 describes the synthesis of (3-acrylamidopropyl) trimethyl ammonium chloride (APTAC) homopolymers of varying molecular weights, using a radical polymerisation reaction. According to the described method, APTAC monomer is polymerised in an aqueous medium by a discontinuous adiabatic process using an azo or persulfate radical initiator. The APTAC homopolymers so obtained have molecular weights ranging from about 100,000 g/mol to about 1,000,000 g/mol. The molecular weight can be determined by using standard analytical measurements, such as size exclusion chromatography (SEC).

A polymer (ii) suitable for use in the invention is commercially available from Ashland, Inc. as N-DurHance™ A-1000 Conditioning Polymer (supplied as a 20% a.i. aqueous solution of the polymer (ii)).

In a typical composition according to the invention the level of polymer (ii) (per se as active ingredient) generally ranges from about 0.1 to about 2% and preferably ranges from about 0.2 to about 1.5% (by weight based on the total weight of the composition).

The composition of the invention includes one or more aliphatic carboxylic acids (iii) having a molecular weight of 175 g/mol or less.

Suitable aliphatic carboxylic acids (iii) for use in the invention have a molecular weight ranging from about 75 to about 160 g/mol, and include lactic acid (90.08 g/mol), glycolic acid (76.05 g/mol), malonic acid (104.06 g/mol), succinic acid (118.09 g/mol), malic acid (134.09 g/mol), and tartaric acid (150.09 g/mol).

Mixtures of any of the above-described materials may also be suitable.

Preferred aliphatic carboxylic acids (iii) for use in the invention contain at least one hydroxy group.

Most preferably the aliphatic carboxylic acid (iii) is tartaric acid.

In a typical composition according to the invention the level of aliphatic carboxylic acids (iii) generally ranges from about 0.1 to about 2% and preferably ranges from 0.5 to 1.5% (by weight based on the total weight of the composition).

A preferred composition according to the invention includes tartaric acid at a level ranging from 0.5 to 1.5% (by weight based on the total weight of the composition).

### Product Form

Hair treatment compositions of the present invention are intended for topical application to the hair and may for example be formulated as lotions, creams, serums, sprays, mousses, waxes or gels.

Hair treatment compositions of the invention may be rinse-off products or leave-on products.

Rinse-off products are intended to be substantially rinsed off the hair of the user with water after use, such as shampoos. Rinse-off products also include conditioners which are intended for application to the hair post-wash and which may be rinsed immediately after application or (for more intensive conditioning), left on the hair for up to 2 hours, e.g. 5 minutes to 2 hours.

Leave-on products are intended not to be rinsed off the hair of the user immediately after use (i.e. within at least the first 2 hours, preferably at least four hours, after application of the product). Leave-on products include for example lotions, creams and serums for use in-between washes, and leave-on conditioners for application to the hair post-wash.

Preferred product forms are shampoos and conditioners.

Shampoos and conditioners according to the invention will generally comprise from about 50 to about 98% water, preferably from about 60 to about 90% water (by weight based on the total weight of the composition).

Other organic solvents may also be present, such as lower alkyl alcohols and polyhydric alcohols. Examples of lower alkyl alcohols include C1 to C6 monohydric alcohols such as ethanol and isopropanol. Examples of polyhydric alcohols include propylene glycol, hexylene glycol, glycerol and propanediol. Mixtures of any of the above-described organic solvents may also be used.

A shampoo composition according to the invention will typically include one or more anionic surfactants which are cosmetically acceptable and suitable for topical application to the hair.

Typical anionic surfactants for use in the invention include those surface active agents which contain an organic hydrophobic group with from 8 to 14 carbon atoms, preferably from 10 to 14 carbon atoms in their molecular structure; and at least one water-solubilising group which is preferably selected from sulfate, sulfonate, sarcosinate and isethionate.

Specific examples of such anionic surfactants include ammonium lauryl sulfate, ammonium laureth sulfate, trimethylamine lauryl sulfate, trimethylamine laureth sulfate, triethanolamine lauryl sulfate, trimethylethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, ammonium cocoyl sulfate, ammonium lauroyl sulfate, sodium cocoyl sulfate, sodium lauryl sulfate, potassium cocoyl sulfate, potassium lauryl sulfate, monoethanolamine cocoyl sulfate, monoethanolamine lauryl sulfate, sodium tridecyl benzene sulfonate, sodium dodecyl benzene sulfonate, sodium cocoyl isethionate and mixtures thereof.

A preferred class of anionic surfactants for use in the invention are alkyl ether sulfates of general formula:

R—O—(CH₂CH₂-O)ₙ—O₃⁻M⁺

in which R is a straight or branched chain alkyl group having 10 to 14 carbon atoms, n is a number that represents the average degree of ethoxylation and ranges from 1 to 5, preferably from 1 to 3.5, and M is an alkali metal, ammonium or alkanolammonium cation, preferably sodium, potassium, monoethanolammonium or triethanolammonium, or a mixture thereof.

Specific examples of such preferred anionic surfactants include the sodium, potassium, ammonium or ethanolamine salts of C₁₀ to C₁₂ alkyl sulfates and C₁₀ to C₁₂ alkyl ether sulfates (for example sodium lauryl ether sulfate (nEO) in which n ranges from 1 to 3.5).

The level of anionic surfactant will generally range from about 5 to 26%, and preferably ranges from 10 to 16% (by weight based on the total weight of the composition).

A shampoo composition according to the invention can optionally include co-surfactants, to help impart aesthetic, physical or cleansing properties to the composition.

A preferred type of co-surfactant is an amphoteric surfactant. Suitable amphoteric surfactants are betaines, such as those having the general formula R(CH₃)₂N⁺CH₂COO⁻, where R is an alkyl or alkylamidoalkyl group, the alkyl group preferably having 10 to 16 carbon atoms. Particularly suitable betaines are oleyl betaine, caprylamidopropyl betaine, lauramidopropyl betaine, isostearylamidopropyl betaine, and cocoamidopropyl betaine.

Mixtures of any of the above-described materials may also be used.

When included, the total level of amphoteric surfactant is generally from 0.1% to 20%, preferably from 1% to 10%, more preferably from 1% to 5% (by weight based on the total weight of the composition).

A shampoo composition according to the invention may include one or more cationic deposition polymers which are selected from cationic polygalactomannans having a mean charge density at pH 7 from 0.2 to 2 meq/g, preferably from 0.5 to 1.8 meq/g. Such polymers may serve to enhance the delivery of conditioning agents from the composition to the skin and/or hair surface during consumer use, thereby improving the conditioning benefits obtained.

The term “charge density” in the context of this invention refers to the ratio of the number of positive charges on a monomeric unit of which a polymer is comprised to the molecular weight of the monomeric unit. The charge density multiplied by the polymer molecular weight determines the number of positively charged sites on a given polymer chain. The charge density is suitably determined via the Kjeldahl method as described in the US Pharmacopoeia under chemical tests for nitrogen determination.

Preferred cationic polygalactomannans for use in the invention include guar hydroxypropyltrimethylammonium chlorides.

Guar hydroxypropyltrimethylammonium chlorides for use in the invention are generally comprised of a nonionic guar gum backbone that is functionalized with ether-linked 2-hydroxypropyltrimethylammonium chloride groups, and are typically prepared by the reaction of guar gum with N-(3-chloro-2-hydroxypropyl) trimethylammonium chloride.

Cationic polygalactomannans for use in the invention (preferably guar hydroxypropyltrimethylammonium chlorides) generally have an average molecular weight (weight average molecular mass (Mw) determined by size exclusion chromatography) in the range 500,000 to 3 million g/mol, more preferably 800,000 to 2.5 million g/mol.

Cationic polygalactomannans for use in the invention are commercially available from Rhodia as JAGUAR® C13S, JAGUAR® C14 and JAGUAR® C17.

Mixtures of any of the above-described materials may also be used.

When included, the total level of of cationic polygalactomannan will generally range from 0.05 to 0.25%, and preferably ranges from 0.15 to 0.2% (by weight based on the total weight of the composition).

A shampoo composition according to the invention may include one or more suspending agents. Suitable suspending agents include polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives.

Mixtures of any of the above suspending agents may be used. Preferred is a mixture of cross-linked polymer of acrylic acid and crystalline long chain acyl derivative.

When included, the total level of suspending agent is generally 0.1 to 10%, preferably from 0.5 to 6%, more preferably from 0.9 to 4% (by weight based on the total weight of the composition).

A conditioner according to the invention is preferably rinse-off as defined above, and will typically include a conditioning gel phase, which may be generally characterized as a gel (L⋅) surfactant mesophase consisting of surfactant bilayers. Such a conditioning gel phase may be formed from a cationic surfactant and a fatty alcohol. Typically these components are heated to form a mixture, which is cooled under shear to room temperature. The mixture undergoes a number of phase transitions during cooling, normally resulting in a gel (L⋅) surfactant mesophase consisting of surfactant bilayers.

Examples of suitable cationic surfactants which are useful for forming the conditioning gel phase include quaternary ammonium cationic surfactants corresponding to the following general formula:

[N(R1)(R2)(R3)(R4)]+(X)—

in which R1, R2, R3, and R4 are each independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a salt-forming anion such as those selected from halide, (e.g. chloride, bromide), acetate, citrate, lactate, glycolate, phosphate nitrate, sulfate, and alkylsulfate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated.

Specific examples of such quaternary ammonium cationic surfactants of the above general formula are cetyltrimethylammonium chloride, behenyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, dipalmitoylethyldimethylammonium chloride, PEG-2 oleylammonium chloride and salts of these, where the chloride is replaced by other halide (e.g., bromide), acetate, citrate, lactate, glycolate, phosphate nitrate, sulfate, or alkylsulfate.

In a preferred class of cationic surfactant of the above general formula, R1 is a C₁₆ to C₂₂ saturated or unsaturated, preferably saturated, alkyl chain and R2, R3 and R4 are each independently selected from CH₃ and CH₂CH₂OH, preferably CH₃.

Specific examples of such preferred quaternary ammonium cationic surfactants for use in forming the conditioning gel phase are cetyltrimethylammonium chloride (CTAC), behenyltrimethylammonium chloride (BTAC) and mixtures thereof.

Mixtures of any of the above-described cationic surfactants may also be suitable. The level of cationic surfactant will generally range from about 0.2 to 5% and more preferably ranges from 0.25 to 4% (by weight based on the total weight of the composition).

The fatty alcohol can be used as a single compound or as a blend or mixture of at least two fatty alcohols.

Suitable fatty alcohols which are useful for forming the conditioning gel phase have a melting point of 25° C. or higher. Generally the melting point ranges from 25° C. up to 90° C., preferably from 40° C. up to 70° C. and more preferably from 50° C. up to about 65° C. When a blend or mixture of fatty alcohols is used, the melting point means the melting point of the blend or mixture.

Examples of suitable fatty alcohols which are useful for forming the conditioning gel phase include fatty alcohols having the general formula R—OH, where R is an aliphatic carbon chain. Preferably R is a saturated aliphatic carbon chain comprising from 8 to 30 carbon atoms, more preferably from 14 to 30 carbon atoms and most preferably from 16 to 22 carbon atoms.

R can contain, in addition to carbon and hydrogen atoms, ether linkages, and other groups such as amino groups.

Most preferably, the fatty alcohol has the general formula CH₃(CH₂)ₙOH, where n is an integer from 7 to 29, preferably from 15 to 21.

Specific examples of suitable fatty alcohols are cetyl alcohol, stearyl alcohol, behenyl alcohol, and mixtures thereof. Cetyl alcohol, stearyl alcohol and mixtures thereof are particularly preferred.

Mixtures of any of the above-described fatty alcohols may also be suitable.

The level of fatty alcohol will generally range from about 0.1 to 8% and more preferably ranges from 0.3 to 6% (by weight based on the total weight of the composition).

The weight ratio of cationic surfactant to fatty alcohol is suitably from 1:1 to 1:10, preferably from 1:1.5 to 1:8, optimally from 1:2 to 1:5.

A composition of the invention, such as a shampoo or conditioner, may also include emulsified droplets of non-volatile silicone having a mean droplet diameter (D3,2) of 1 micrometre or less. Preferably the mean droplet diameter (D3,2) is 1 micrometre or less, more preferably 0.5 micrometre or less, and most preferably 0.25 micrometre or less.

A suitable method for measuring the mean droplet diameter (D3,2) is by laser light scattering using an instrument such as a Malvern Mastersizer.

The term “non-volatile silicone” in the context of this invention means a silicone with a vapour pressure of less than 1000 Pa at 25° C.

Suitable silicones for use in the invention include polydiorganosiloxanes, in particular polydimethylsiloxanes (dimethicones), polydimethyl siloxanes having hydroxyl end groups (dimethiconols), and amino-functional polydimethylsiloxanes (amodimethicones).

Suitable silicones preferably have a molecular weight of greater than 100,000 and more preferably a molecular weight of greater than 250,000.

All molecular weights as used herein are weight average molecular weights, unless otherwise specified.

Suitable silicones preferably have a kinematic viscosity of greater than 50,000 cS (mm²·s⁻¹) and more preferably a kinematic viscosity of greater than 500,000 cS (mm²·s⁻¹). Silicone kinematic viscosities in the context of this invention are measured at 25° C. and can be measured by means of a glass capillary viscometer as set out further in Dow Corning Corporate Test Method CTM004 Jul. 20, 1970.

Suitable silicones for use in the invention are available as pre-formed silicone emulsions from suppliers such as Dow Corning and GE Silicones. The use of such pre-formed silicone emulsions is preferred for ease of processing and control of silicone particle size. Such pre-formed silicone emulsions will typically additionally comprise a suitable emulsifier, and may be prepared by a chemical emulsification process such as emulsion polymerisation, or by mechanical emulsification using a high shear mixer. Pre-formed silicone emulsions having a mean droplet diameter (D3,2) of less than 0.15 micrometers are generally termed microemulsions.

Examples of suitable pre-formed silicone emulsions include emulsions DC2-1766, DC2-1784, DC-1785, DC-1786, DC-1788, DC-1310, DC-7123 and microemulsions DC2-1865 and DC2-1870, all available from Dow Corning. These are all emulsions/microemulsions of dimethiconol. Also suitable are amodimethicone emulsions such as DC939 (from Dow Corning) and SME253 (from GE Silicones).

Mixtures of any of the above-described silicone emulsions may also be used.

When included, the amount of emulsified, non-volatile silicone may suitably range from 0.05 to 10%, preferably from 0.2 to 8% (by total weight silicone based on the total weight of the composition).

A composition of the invention, such as a shampoo or conditioner, may contain further optional ingredients to enhance performance and/or consumer acceptability. Examples of such ingredients include fragrance, dyes and pigments and pH adjusting agents. Each of these ingredients will be present in an amount effective to accomplish its purpose. Generally, these optional ingredients are included individually at a level of up to 5% by weight based on the total weight of the composition.

The invention will be further illustrated by the following, non-limiting Examples.

### EXAMPLES

Hair cleansing shampoo formulations were prepared, having ingredients as shown in Table 1. Comparative Examples (not according to the invention) are indicated by letter; Examples according to the invention are indicated by number.

TABLE 1

| | Active % | | | | |
|---|---|---|---|---|---|
| | in raw | Active % in shampoo formulation | | | |
| Ingredient | mat. | Ex. A | Ex. 1 | Ex. B | Ex. 2 |
| Sodium laureth sulfate | 70 | 12 | 12 | 12 | 12 |
| (1EO) | | | | | |
| Cocamidopropyl | 30 | 1.6 | 1.6 | 1.6 | 1.6 |
| betaine | | | | | |
| Carbomer | 100 | 0.4 | 0.4 | 0.4 | 0.4 |
| Ethylene glycol | 25 | 0.625 | 0.625 | 0.625 | 0.625 |
| distearate | | | | | |
| Dimethiconol* | 50 | 1 | 1 | — | — |
| Sodium chloride | 100 | 0.4 | 2.1 | 0.4 | 2.1 |
| Citric acid | 100 | 0.5 | — | 0.5 | — |
| Sodium hydroxide | 50 | 0.15 | 0.45 | 0.15 | 0.45 |
| Guar hydroxypropyl- | 100 | 0.2 | 0.2 | 0.2 | 0.2 |
| trimonium chloride | | | | | |
| Sodium benzoate | 100 | 0.5 | 0.5 | 0.5 | 0.5 |
| Disodium EDTA | 100 | 0.05 | 0.05 | 0.05 | 0.05 |
| Lytol ® mineral oil | 100 | — | 1 | — | 1 |
| Tartaric acid | 100 | — | 1 | — | 1 |
| PolyAPTAC | 20 | — | 1 | — | 1 |
| homopolymer** | | | | | |
| Mica | 100 | 0.2 | 0.2 | 0.2 | 0.2 |
| Perfume, preservative, | | to 100% | to 100% | to 100% | to 100% |
| water | | | | | |

*DOW CORNING ® 1788 silicone emulsion
**N-DurHance ™ A-1000 conditioning polymer (ex Ashland)

All formulations are specified at viscosity from 4000 to 6000 mPa·s measured using a Brookfield V2 viscometer (spindle RTV5, 1 minute, 20 rpm) at 30° C.

### Cuticle Abrasion Method

The formulations described in Table 1 were used to treat test twice bleached test switches of hair. The treated switches were evaluated by a cuticle abrasion method which assesses hair damage by quantifying the susceptibility for cuticle to be removed from the fibre surface in water.

### Protocol

5g straight dark brown European (DBE) hair switches were washed twice for 30 seconds, followed by a 30 second rinse using the test formulation. The rinsed switches were then dried at 50° C. in a drying cabinet, cut into small portions (1-2 cm) and placed into a Waring blender. Distilled water (200 mls) was added and the mixture agitated at high speed for one minute. The liquor containing abraded cuticle was filtered through a 425 μM mesh sieve to remove hair fragments which were then discarded. The liquor was then filtered through a Buchner funnel filtration system on a pre weighed filter paper. The filter paper was allowed to dry, weighed again and the mass of cuticle calculated

### Results

| Test formulation | Mean mg/g |
|---|---|
| Example A | 1.08 |
| Example 1 | 0.81 |
| Example B | 1.18 |
| Example 2 | 0.85 |

### Differential Scanning Calorimetry (DSC)

The formulations described in Table 1 were used to treat twice bleached test switches of hair. The treated switches were evaluated by DSC.

### Protocol

Approximately 5-7 mg of shavings were collected from the switches treated with the test formulation and placed into individual steel HPDSC crucibles before adding 50·l of water and sealing them.

The previously-recorded mass of samples was entered into the software to allow the heat capacity of the sample to be calculated by the instrument. Samples were heated from 100-180° C. at a rate of 5° C. per minute.

When a run was complete, the endotherm peak for hair denaturation was identified and the peak integrated. The calculated denaturation temperature and enthalpy was recorded and averages calculated.

### Results 1

| Test formulation | Mean Td (° C.) |
|---|---|
| Example A | 146.23 |
| Example 1 | 147.68 |
| Example B | 146.23 |
| Example 2 | 149.60 |

It can be seen from the results that the hair fibres treated with Examples 1 or 2 according to the invention have improved surface integrity (as evidenced by reduced cuticle abrasion) as well as improved core strength (as evidenced by increased denaturation temperature).

This shows that the Examples according to the invention are able to effect strengthening and/or repair of damaged hair.

### Results 2

| Test formulation | Mean Td (° C.) |
|---|---|
| Clean hair without treatment | 146.31 |
| 1% aconitic acid | 145.83 |
| 1% Tartaric acid | 149.54 |
| 1% Lactic acid | 146.67 |

Data in Results 2 are generated from a separate round of DSC experiment, wherein the formulation comprises 1% of different carboxylic acids by weight of the total composition. Analysis shows 1% tartaric acid is better than 1% lactic acid at 99% confidence level. 1% aconitic acid (Mw=174 g/mol) which is not according to the invention, does not increase the denature temperature of hair. From the data, it appears the formulation comprising tartaric acid according to the invention provides the best repair/strength to damaged hair.

1. A hair treatment composition for the strengthening and/or repair of damaged hair, the composition comprising, in an aqueous continuous phase:
(i) one or more oily liquid hair conditioning agents selected from solubilized oily liquid hydrocarbons and mixtures thereof;
(ii) a hair substantive cationic conditioning polymer which is a homopolymer of (3-acrylamidopropyl) trimethyl ammonium chloride, and
(iii) an aliphatic carboxylic acid having a molecular weight of 175 g/mol or less, which is a tartaric acid.

2. The composition according to claim 1, in which the oily liquid hair conditioning agent (i) is light mineral oil having a kinematic viscosity of 3 to 5 cS at 40° C.

3. The composition according to claim 3, in which the level of the light mineral oil ranges from 0.5 to 1.5% by weight based on the total weight of the composition.

4. The composition according to claim 1, in which the level of cationic conditioning polymer (ii) ranges from 0.2 to 1.5% by weight based on the total weight of the composition.

5. The composition according to claim 1, in which the level of the tartaric acid ranges from 0.5 to 1.5% by weight based on the total weight of the composition.

6. The composition according to claim 1, which is in the form of a shampoo and comprises from 60 to 90% water and from 10 to 16% anionic surfactant by weight based on the total weight of the composition.

7. The composition according to claim 1, which is in the form of a rinse-off conditioner and comprises from 0.25 to 4% cationic surfactant and from 0.3 to 6% fatty alcohol by weight based on the total weight of the composition.
